(19) United States
(12) Patent Application Publication
(10) Pub. No.: US 2005/0245947 A1
(43) Pub. Date: Nov. 3, 2005

(54) Transvaginal uterine artery occlusion for treatment of uterine leiomyomas

(76) Inventor: Oz Harmanli, Blue Bell, PA (US)

Correspondence Address: PATRICIA A. WENGER, 201 NORTH JACKSON STREET, MEDIA, PA, 19063, US

(21) Appl. No.: 10/837,156

(22) Filed: Apr. 30, 2004

(51) Int. Cl. A61B 17/08

(52) U.S. Cl. 606/157

(57) ABSTRACT

A method for treating uterine leiomyomas, which includes vaginally accessing the cardinal ligament surrounding at least one uterine artery, and occluding the flow of blood through the least one uterine artery. The blood can be occluded by compression. The artery can be compressed by ligating the cardinal ligament. In addition, at least one uterosacral ligament can be ligated. In addition, the artery can be compressed by, clipping, stapling or clamping the cardinal ligament. The blood can also be occluded by coagulating the at least one artery. Coagulation can be done with a laser or a cauterizing device. A system for treating uterine leiomyomas, which includes vaginally accessing the cardinal ligament surrounding at least one uterine artery using a surgical suture passing device to extend a ligature around the ligament, and affixing a ligature around the cardinal ligament to occlude the flow of blood through at least one uterine artery. Also, a system for treating uterine leiomyomas, which includes vaginally accessing the cardinal ligament surrounding at least one uterine artery, providing an absorbable clip and/or staple, and affixing the clip and/or staple around the ligament to occlude the flow of blood through the at least one uterine artery.

### BACKGROUND OF THE INVENTION

Uterine leiomyomas are generally described as benign smooth-muscle tumors, and commonly known as fibroids. A leiomyoma can be located in any portion of the uterus.

Known treatments for uterine leiomyomas include hormonal treatment, uterine artery embolization, myomectomy, and hysterectomy. While these treatments have seen many satisfactory results, each treatment also presents potential risk.

Leiomyomas have been identified as the most common indication for hysterectomy in the United States. Hysterectomy, which includes the surgical removal of the uterus, is a highly invasive procedure.

Uterine artery embolization, while less invasive than hysterectomy, includes the risk of stray pellets affecting the ovaries and causing premature menopause.

The uterine artery laparoscopic closure procedure also is less invasive than hysterectomy. However, this procedure requires exceptional skills, and presents a considerable risk of damage to the ureters due to the proximity of the ureters to the uterine arteries.

The method and system of the current invention presents a novel transvaginal uterine artery occlusion treatment for uterine leiomyomas.

### SUMMARY OF THE INVENTION

A method for treating uterine leiomyomas, which includes vaginally accessing the cardinal ligament surrounding at least one uterine artery, and occluding the flow of blood through the at least one uterine artery. The blood can be occluded by compressing the at least one artery.

The artery can be compressed by ligating the cardinal ligament. In addition, at least one uterosacral ligament can be ligated. The artery can also be compressed by affixing a clip around at least a portion of the cardinal ligament. In addition, the artery can be compressed by stapling or clamping the cardinal ligament.

The blood can also be occluded by coagulating the at least one artery. Coagulation can be done with a laser or a cauterizing device.

The invention includes a method for treating uterine leiomyomas, which includes vaginally accessing the cardinal ligament surrounding at least one uterine artery, and occluding the flow of blood through two uterine arteries.

Further, the invention includes a method for treating uterine leiomyomas, which includes vaginally accessing the cardinal ligament surrounding at least one uterine artery, and occluding the flow of blood through the least one uterine artery, as well as entering the cul-de-sac and the avascular vesicouterine space.

In addition, the invention includes a method for treating uterine leiomyomas that includes vaginally accessing the cardinal ligament surrounding at least one uterine artery, and occluding the flow of blood through the least one uterine artery. In addition, the blood flow through the uterine artery into the uterus after occluding the at least one artery can be measured.

A system for treating uterine leiomyomas is included, which includes vaginal access of the cardinal ligament surrounding at least one uterine artery using a surgical suture passing device to place a ligature around the ligament, and affixing a ligature around the cardinal ligament to occlude the flow of blood through the at least one uterine artery. The system can further include using a surgical suture passing device that has a handle comprised of a thumb and a finger receptacle. Also, the system can include using a surgical suture passing device that has a jaw portion, which is large enough to fit around the cardinal ligament. Further, the system can include using a surgical suture passing device to ligate at least one uterosacral ligament.

Also disclosed is a system for treating uterine leiomyomas, which includes vaginal access of the cardinal ligament surrounding at least one uterine artery, providing an absorbable clip, and affixing the clip around the ligament to occlude the flow of blood through the at least one uterine artery.

Further disclosed is a system for treating uterine leiomyomas, which includes vaginally accessing the cardinal ligament surrounding at least one uterine artery, providing an absorbable staple, and affixing the staple around the ligament to occlude the flow of blood through the at least one uterine artery.

Another embodiment of the invention includes system for treating uterine leiomyomas, which includes vaginally accessing the cardinal ligament surrounding at least one uterine artery, providing a cauterizing device, and using the cauterizing device to occlude the flow of blood through the at least one uterine artery.

A further embodiment of the invention includes system for treating uterine leiomyomas, which includes vaginally accessing the cardinal ligament surrounding at least one uterine artery, providing a laser, and using the laser to occlude the flow of blood through the at least one uterine artery.

### BRIEF DESCRIPTION OF THE DRAWINGS

The accompanying drawings, which are incorporated in, and form a part of, the specification, illustrate the embodiments of the present invention. The drawings, together with the description, serve to explain the principles of the invention.

FIG. 1 is a drawing of a healthy uterus.

FIG. 2 is drawing of a uterus having leiomyomas.

FIG. 3 is drawing of a uterus having leiomyomas, shown after uterine artery blood flow has been occluded.

FIG. 4 is a Doppler ultrasound measurement of uterine artery blood flow before occlusion.

FIG. 5 is a surgical view of a uterus.

FIG. 6 is a surgical view of a uterus.

FIG. 7 is a surgical view of a uterus.

FIG. 8 is a drawing of a uterus indicating occlusion sites.

FIG. 9 is a drawing of a frontal view of a uterus indicating occlusion sites.

FIG. 10 is a cross-sectional view of a cardinal ligament.

FIG. 11 is a cross-sectional view of a cardinal ligament that has been ligated.

FIG. 12 is a surgical view of a uterus utilizing a surgical suture passing device.

FIG. 13 is a surgical view of a uterus utilizing a surgical suture passing device.

FIG. 14 is a surgical view of a uterus utilizing a suture carrier device.

FIG. 15 is a surgical view of a uterus utilizing a suture carrier device.

FIG. 16 is a surgical view of a uterus utilizing clips.

FIG. 17 is a surgical view of a uterus utilizing a clamp.

FIG. 18 is a surgical view of a uterus utilizing a cauterizing device.

FIG. 19 is a surgical view of a uterus utilizing a laser.

FIG. 20 is a Doppler ultrasound measurement of uterine artery blood flow after occlusion.

FIG. 21 is a surgical view of a uterus.

FIG. 22 is an exploded view of a uterus.

FIG. 23 is an enlarged exploded view of a cardinal ligament.

FIG. 24 is an exploded view of a uterus.

FIG. 25 is an enlarged exploded view of a cardinal ligament that has been constricted by a ligature.

FIG. 26 is a surgical view of a uterus utilizing staples.

FIG. 27 is a surgical view of a uterus utilizing a suture carrier device.

FIG. 28 is a surgical view of a uterus utilizing a suture carrier device.

### DETAILED DESCRIPTION OF THE PREFERRED EMBODIMENTS

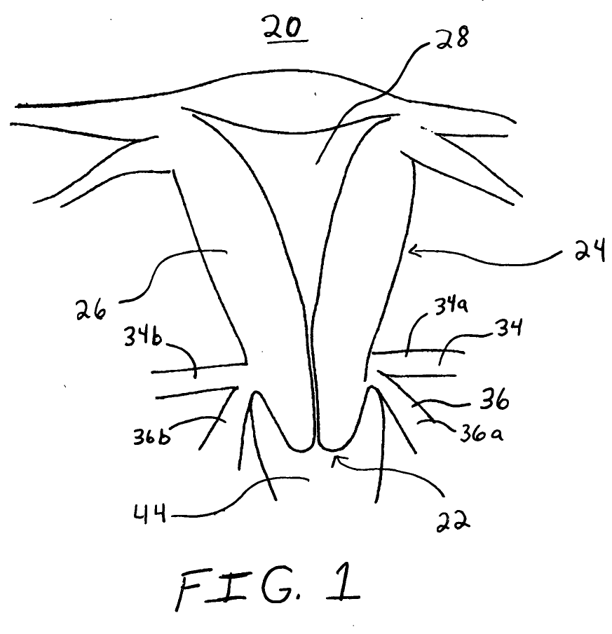

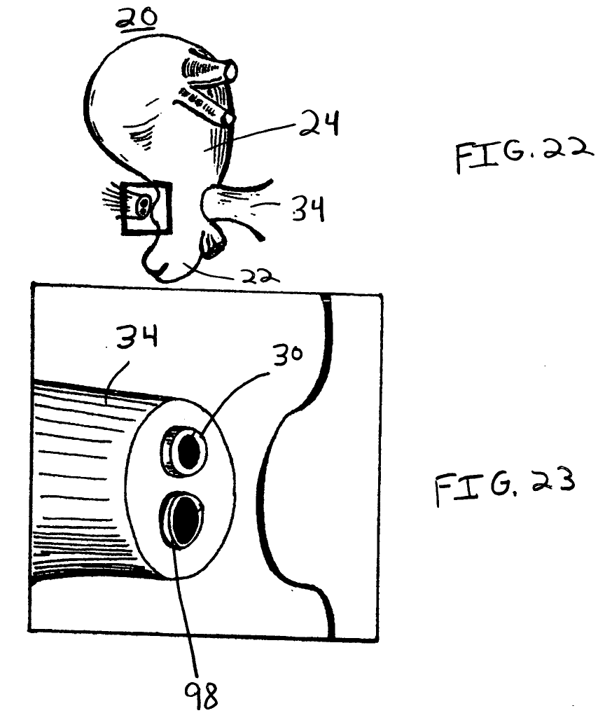

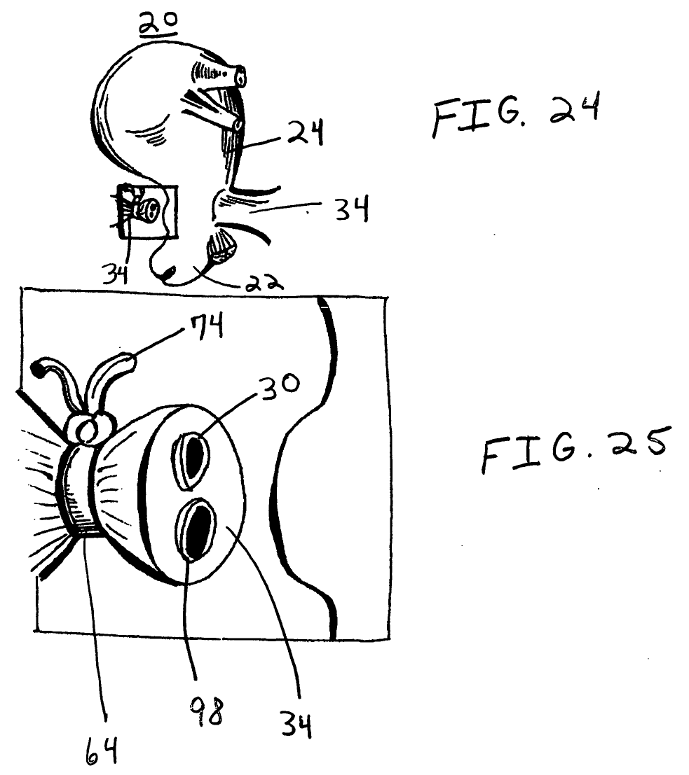

Embodiments of a new method and system for treating uterine leiomyomas 38 are described with reference to the drawings, and in particular to FIGS. 1 through 28, in which like parts are given like reference numerals.

Reference is now made to FIG. 1, which illustrates the uterus 20 of a healthy female. The uterus 20 has a lower portion, which is known as the cervix 22, and an upper portion, known as the corpus 24. Among other structures, the uterus 20 includes uterine walls 26 and a uterine cavity 28. The uterus 20 accesses blood 32, by a number of means. The most direct means is through the uterine arteries 30. Typically a female has two uterine arteries 30; one artery generally on the left side 30a of the uterus 20 and one artery generally on the right side 30b of the uterus 20.

The uterus 20 is supported by means that include two cardinal ligaments 34, one on the left side 34a of the uterus 20 and one on the right side 34b of the uterus 20. In addition, the uterus 20 is supported by two uterosacral ligaments 36, one on the left side 36a of the uterus 20 and one on the right side 36b of the uterus 20. Typically, the uterine artery 30 is surrounded by the cardinal ligament 34 when the artery enters the uterus 20. Generally, the uterosacral ligaments 36 do not contain arteries. However, some branches of a uterine artery may be in the vicinity of a uterosacral ligament 36. It is also possible for the uterosacral ligament 36 and the cardinal ligament 34 to merge into a uterosacral cardinal complex which can attach to the uterus 20 in a merged manner. In addition, a uterosacral ligament 36 could include collateral sources of blood 32 that could flow to the uterus 20.

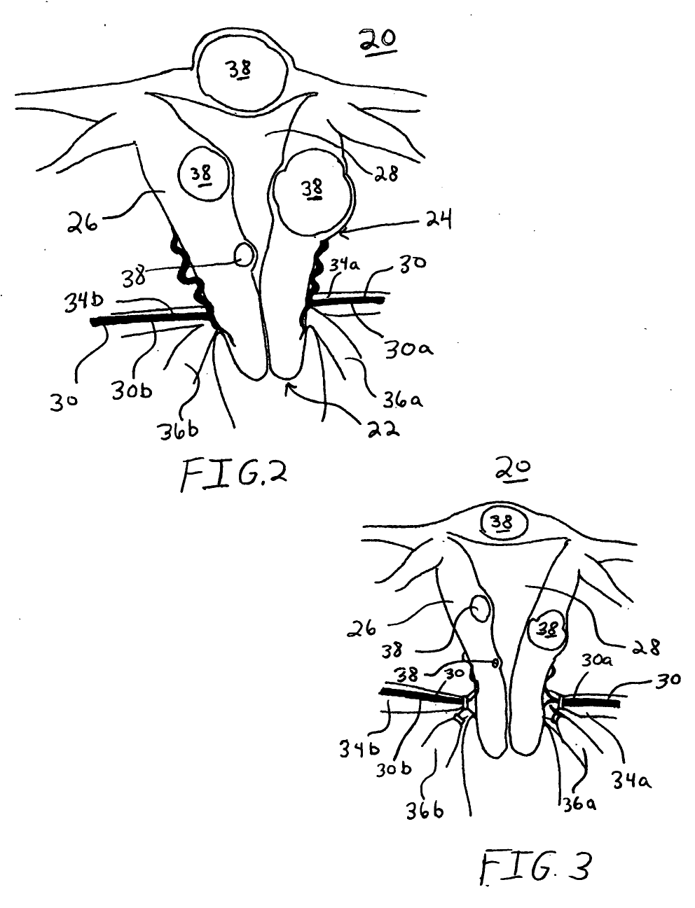

FIG. 2, illustrates a uterus 20 with leiomyomas 38. Leiomyomas 38 are well known in the art, and generally described as benign smooth-muscle tumors. Leiomyomas 38 are also commonly known as fibroids. A leiomyoma 38 can attach to any portion of the uterus 20.

FIG. 3 illustrates leiomyomas 38 after the at least one uterine artery 30 has been occluded. As seen in FIG. 3 in conjunction with FIG. 2, after occlusion of the artery, the size of the leiomyomas 38 is reduced. The lack of blood to the leiomyomas 38 shrink them. In addition, the lack of blood to the uterus 20 can cause the overall size of the uterus 20 to be reduced.

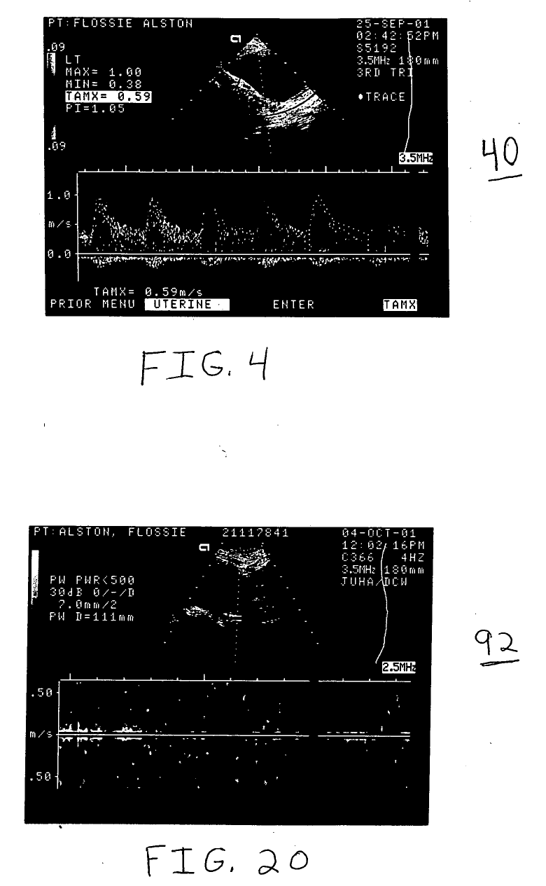

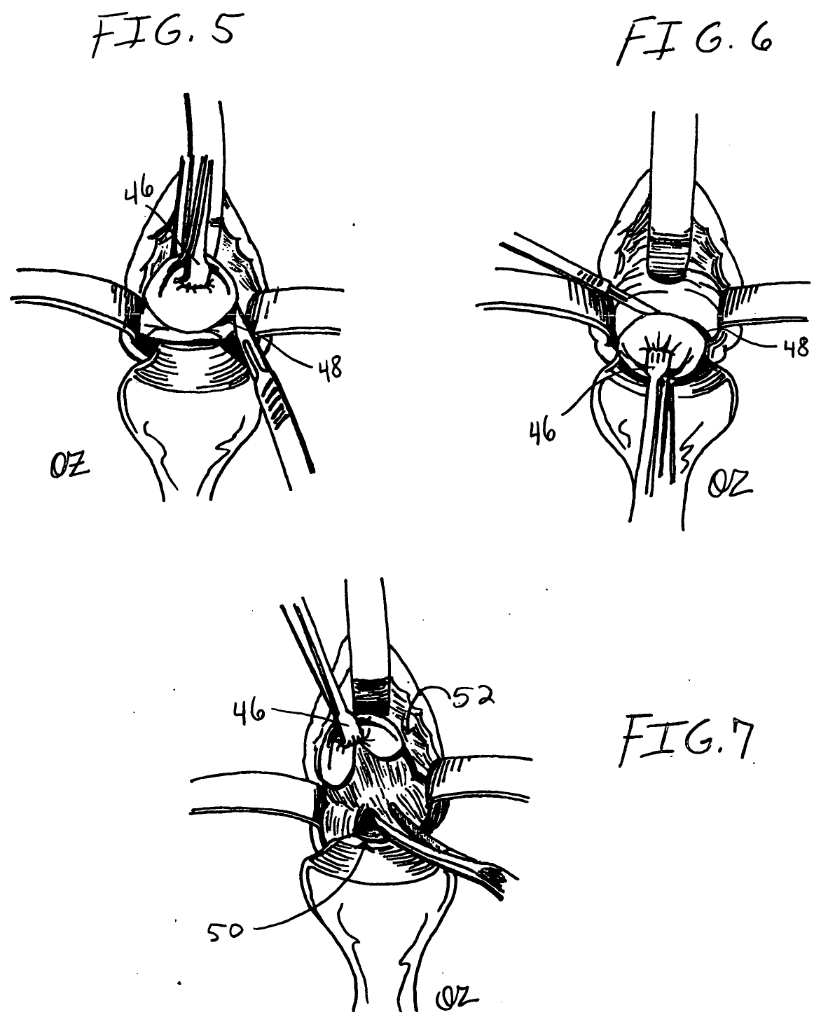

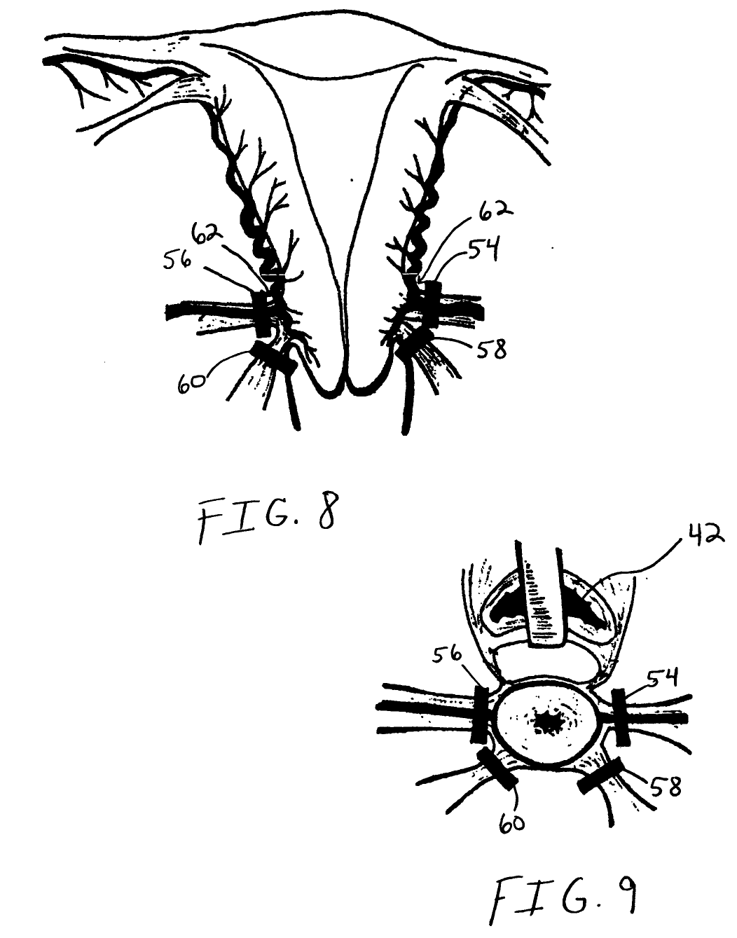

Referring now to FIG. 4 a measurement can be taken of the amount of blood 32 flowing from the uterine artery 30 to the uterus 20. This step is discretionary with the physician. As seen in this Figure, a Doppler ultrasound waveform measurement 40 may be recorded of the blood flow before occlusion. While use of the Doppler waveform has yielded excellent results, it is to be understood that other means currently known or to be discovered are intended to be within the scope of the current invention. In one embodiment, the measurement is taken after the patient is placed in a dorsal lithotomy position and before the bladder 42 is drained. However, it is to be understood that the measurement may be taken at any time before an artery and/or ligament is occluded to provide a baseline.

The initial steps of the method are similar to the initial steps commonly known in the art for performing a hysterectomy. General anesthesia is administered, the vaginal 44 area is prepared and draped, the patient is placed in a dorsal lithotomy position and the patient's bladder 42 is drained. Then, the cervix 22 can be exposed by a weighted speculum and vaginal retractors, and the cervix 22 is grasped with two tenacula 46. Without intending to be limiting, a Lahey tenacula may be used, as well as others known in the art or to be discovered. Thereafter, a circumferential cervical incision 48 is made outside the transformation zone of the uterus 20. FIG. 5 illustrates an incision 48 of the posterior portion of the cervix 22 and FIG. 6 illustrates an incision 48 of the anterior portion of the cervix 22. While the foregoing steps are described to create a circumferential incision 48 in the uterus 20, it is to be understood that these steps of the invention are not intended to be limiting. Other methods currently known in the art or to be discovered to prepare a patient and/or to enable vaginal access to the uterus 20 whereby a circumferential incision in the uterus 20 may be made are intended to be within the scope and intention of the current invention.

During the initial steps, it is also preferable to retract the bladder 42 away from the uterus 20. This will cause the ureters to be pulled away from the uterus 20, where they will be less likely to be impacted and or damaged by the procedure.

Next, the cul-de-sac 50 and avascular vesicouterine space 52 are entered. The cul-de-sac 50 is illustrated in FIG. 7. The avascular vesicouterine space 52 is illustrated in FIG. 28. The order in which the cul-de-sac 50 and avascular vesicouterine space 52 are entered is discretionary with the physician. As known in the art, entering the cul-de-sac 50 and avascular vesicouterine space 52 can include separating the uterus 20 from the abdomen by incising or cutting through the vaginal mucosa, connective tissue and/or peritoneal layer. Excellent results have been achieved by sharply entering the cul-de-sac 50 and avascular vesicouterine space 52 by Mayo scissors. However, it is to be understood that other means currently known or to be discovered for entering the cul-de-sac 50 and avascular vesicouterine space 52 are intended to be within the scope of this invention.

Optionally, the weighted speculum can then be replaced with a longer, less obtrusive speculum to enable complete visualization of the uterosacral ligaments 36.

Next, blood sources flowing through at least one uterine artery and/or one uterosacral ligament 36 to the uterus 20 are occluded. The occlusion can be accomplished by any number of ways, which are later described. In addition, the techniques for occluding the uterine artery and blood 32 sources in the uterosacral ligament 36 are similar. Generally, a uterine artery 30 can be occluded while located in the cardinal ligament 34 or after the artery is dissected away from the ligament. However, occluding the uterine artery 30 while it is located in the cardinal ligament 34 is preferable. Generally, the blood 32 sources flowing through the uterosacral ligament 36 are collateral sources that might include a branch of the uterine artery. Occlusion of the blood 32 sources through the uterosacral ligament 36 generally will occur while the blood source is located in the ligament.

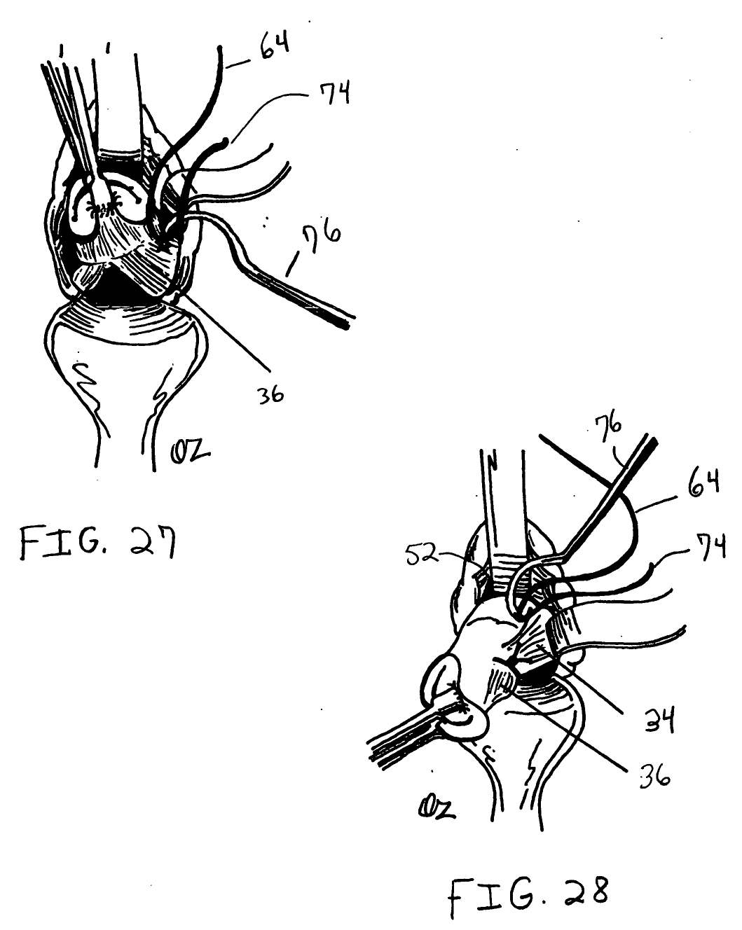

It is to be noted that occlusion of the at least one uterosacral ligament 36 generally is optional because the ligament is not a main source of blood 32 to the uterus 20. However, excellent results have been achieved by occluding the blood flowing through both uterosacral ligaments 36 along with occluding the blood flowing through both uterine arteries 30. It is discretionary with the physician whether to occlude at least one uterosacral ligament 36. By way of example, and not intending to be limiting, a uterosacral ligament 36 might be occluded because it can be a collateral source of blood 32 to the uterus 20. In addition, as illustrated in FIG. 27, in some instances distinguishing between the uterosacral ligament 36 and cardinal ligament 34 might be difficult due to the specific anatomy of the patient. Also, in some instances, the uterosacral ligament 36 and the cardinal ligament 34 merge near their insertion site with the uterus 20 at a uterosacral cardinal complex.

In addition, while occluding both uterine arteries 30 is recommended, there may be any number of reasons why occluding only one of the uterine arteries 30 could occur, while still obtaining desirable benefits. Similarly, there may be any number of reasons why occluding only one of the uterosacral ligaments 36 could occur, while still obtaining desirable benefits. By way of example and not intending to be limiting, a female patient could in rare cases possess only one uterosacral ligament 36 and/or only one cardinal ligament 34 due to genetic reasons or prior injury. In addition, there could be any number of reasons that the physician might decide to occlude only one of the uterosacral ligaments 36s and/or uterine arteries 30, which could include, but would not be limited to, injury, expediency and the like.

The order in which the at least one uterine artery 30 and optionally the blood sources in the uterosacral ligament 36 are occluded is discretionary with the physician. By way of example and not intending to be limiting, if all the uterine arteries 30 and the uterosacral ligaments 36 are going to be occluded, the physician might occlude the at least one uterosacral ligament 36 first because it is closer to the cervical incision 48 than the uterine artery 30. Alternatively, the physician might want to occlude the at least one uterine artery 30 first because the uterine arteries 30 are a main source of blood to the uterus 20. Thereafter, the physician might take a measurement of the blood flow to the uterus 20 before deciding whether to also occlude the at least one uterosacral ligament 36. Any number of factors could influence the physician's choice relating to the order of occlusion. Also, in the preferred case where both of the uterine arteries 30 are occluded the order of occlusion in relation to left and right arteries is discretionary. Similarly, where both uterosacral ligaments 36 are occluded, the order of occlusion in relation to left and right ligaments is discretionary.

The uterine artery 30 and the blood source flowing through the uterosacral ligament 36 may be occluded by any technique to stop or reduce the flow of blood to the uterus 20. Preferably, the blood flow will be totally stopped. In addition, as previously described, preferably, both uterine arteries 30 will be occluded. It is to be understood that any manner currently known or to be discovered by which the blood flow through the at least one uterine artery 30 to the uterus 20 can be occluded is intended to be included within the scope of the present invention. Similarly, it is to be understood that any manner currently known or to be discovered by which the blood flow through the at least one uterosacral ligament 36 to the uterus 20 can be occluded is intended to be included within the scope of the present invention.

Reference is now made to FIGS. 8 and 9, which indicate preferred occlusion points. The occlusion points are the general locations of the part of the ligament and/or artery that is manipulated to create the occlusion of the blood flow. The manipulation can include any technique known or to be discovered that will occlude the blood flow. Techniques can include, but are not limited to, compression, coagulation, blockage and the like. In the preferred embodiment, as illustrated in these figures, the occlusion points are located on the ligament and/or artery immediately lateral to the isthmic 62 portion of the uterus 20. The occlusion points could also be referred to as being immediately lateral to the insertion site of the ligament to the uterus 20. Now specifically referring to the Figures, occlusion point 54 is on the left cardinal ligament 34, occlusion point 56 is on the right cardinal ligament 34, occlusion point 58 is on the left uterosacral ligament 36 and occlusion point 60 is on the right uterosacral ligament 36. However, if necessary and/or desired the occlusion point can be located at other places along the at least one cardinal ligament 34 without departing from the intent and scope of the current invention. When the occlusion point in not located immediately lateral to the isthmic 62 portion it is important to avoid impacting or damaging the ureter, which is located in the vicinity.

The uterine artery 30 may be occluded by compression of the artery. Any manner currently known or to be discovered by which the uterine artery 30 may be occluded by compression is intended to be within the scope of the present invention. It is to be noted that the uterine artery 30 is generally located in the cardinal ligament 34 when it enters the uterus 20. The compression of the artery can be accomplished by compressing the ligament, which causes the artery therein to compress, or by dissecting the artery away from the ligament and compressing just the artery. Compression of the ligament and thereby the artery located therein is preferred, as this embodiment generally will be less invasive than dissecting the artery from the ligament.

Similarly, the blood flowing through the uterosacral ligament 36 may be occluded by compression of the ligament by the same techniques described in relation to the uterine artery. In addition, it is to be understood that any manner currently known or to be discovered by which the blood flowing through the ligament may be occluded by compression is intended to be within the scope of the present invention.

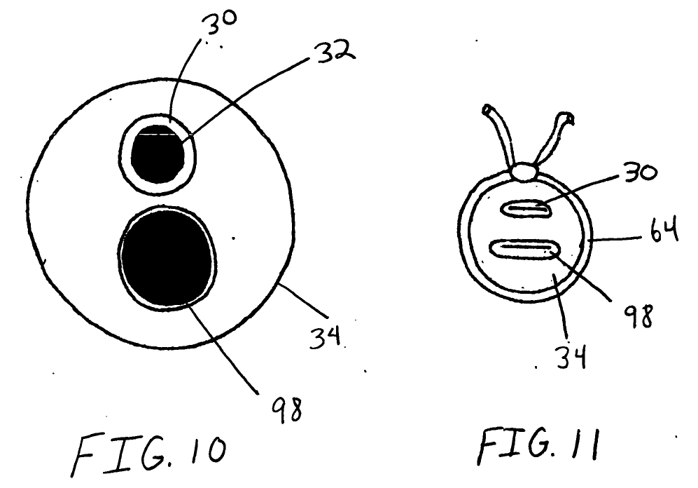

In FIGS. 10, 11, 13, 15, 24, 25 and 28, illustrations are found of an embodiment of the invention in which a ligature in the form of a suture 64 is passed around at least one cardinal ligament 34 and tied securely. The constriction of the ligament causes the uterine artery 30 that is located within the ligament to be likewise constricted. Thereby blood flow to the uterus 20 through the arteries will be occluded.

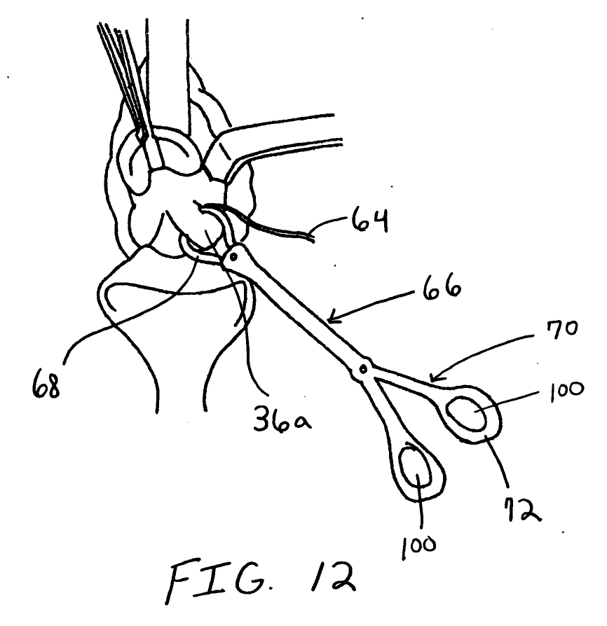

Specific reference is now made to FIGS. 12, 14 and 27, which illustrate embodiments of the invention in which ligatures in the form of sutures 64 are passed around at least one uterosacral ligament 36 and tied securely. The ligature should be tied tightly enough to cause a constriction of the ligament. The constriction of the ligament causes any blood vessels and/or branches of arteries that are located within the ligament to be likewise constricted. Thereby blood flow to the uterus 20 through the vessels and/or branches will be occluded. It is to be understood that the blood 32 flowing through the uterosacral ligament 36 can be occluded by sutures 64 using the same techniques and devices described in relation to occluding the uterine artery.

It is to be noted that for all embodiments that use ligatures in the form of a suture 64, the suture 64 optionally can be absorbable into the tissue over time. However, non-absorbable sutures 64 also can be used.

In this step it is important to make sure that the uterine artery 30 is included within the ligature. As specifically seen in FIGS. 10, 11, and 22 through 25, the securely tied ligature causes the cardinal ligament 34 to constrict along with the uterine artery 30 located within the ligament. The ligament also causes the vein 98 in the cardinal ligament 34 to constrict.

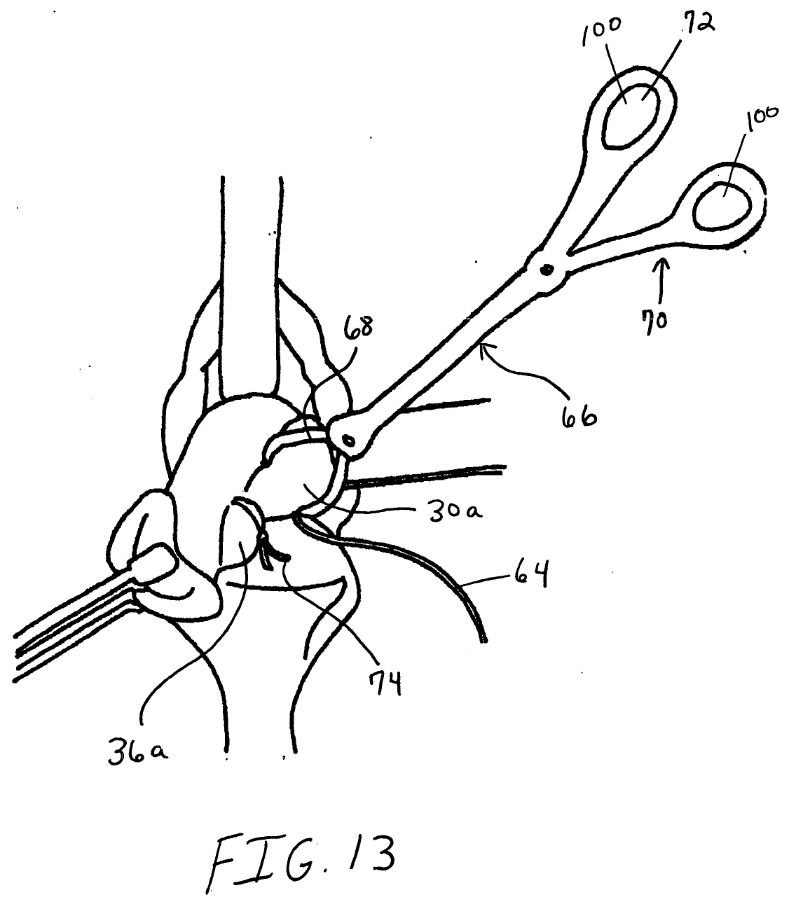

In the embodiments illustrated in FIGS. 12 and 13 a surgical suture passing device 66 is used to position the ligature around the ligament. Surgical suture passing devices 66 are well known in the art. Such as device is described in published U.S. Patent Application No. 20030023250 by Watschke et al, and assigned to AMS Research Corporation (referred to herein as the “AMS suture 64 passing device 66”). The U.S. Patent Application No. 20030023250 is incorporated herein by reference as though set forth in full.

In the current invention, a version of a suture passing device 66 is illustrated in FIGS. 12 and 13. The device includes a set of jaws 68 that are large enough to encircle the size of the cardinal ligament 34 immediately lateral to the isthmic 62 portion of the uterus 20 and the size of the uterosacral ligament 36 near its insertion site with the uterus 20. As a result, the suture passing device 66 illustrated might include a modification of an AMS suture passing device 66 wherein the jaws 68 of the AMS suture passing device 66 may be elongated and/or widened. In addition, as illustrated in the Figures, an optional modification of the AMS suture passing device 66 could be to change the handle 70 to include a more ergonomic handle 70 for the occlusion method of the current invention. Such a modification could include providing a handle 70 with a scissors-like grip 72, which can include receptacles 100 for the user's thumb and finger.

The functioning of the AMS suture passing device 66 is clearly described in U.S. Patent Application No. 20030023250. In the method of the current invention, as illustrated in FIG. 13, the jaws 68 of the suture passing device 66 are placed around the cardinal ligament 34 and the suture 64 is passed from one side of the ligament to the other. At this point, the loose ends 74 of the suture 64 may be grasped, pulled securely around the ligament, and securely tied. As illustrated in FIG. 12, the suture passing device 66 can be similarly utilized to pass the suture 64 around the uterosacral ligament 36.

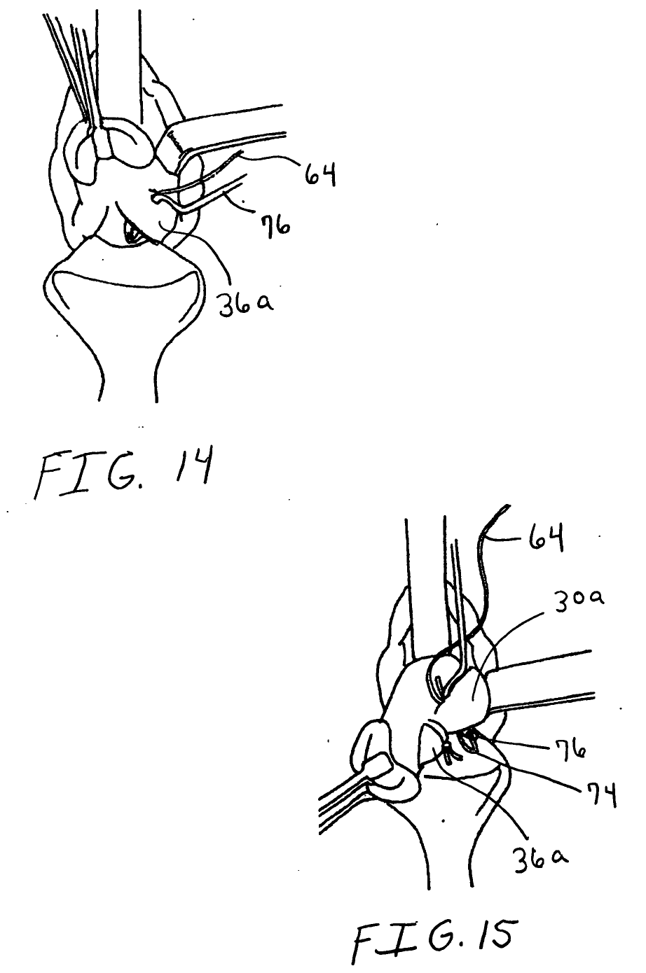

Referring now to FIGS. 14 and 15, a suture carrier device 76, such as but not limited to, a Nichols-Dechamps suture carrier, which is well known in the art, could be used to guide the suture 64 around the cardinal ligament 34. As seen in FIGS. 14 and 27, the suture carrier device 76 can be passed through tissue or part of the ligament and extended around the ligament to a point where the loose end 74 of the suture 64 can be grasped. Alternatively, as illustrated in FIG. 15, if access can be achieved, the suture carrier can pass from one side of the ligament, and around the back of the ligament to a point where the loose end 74 of the suture 64 can be grasped. At this point, the loose ends 74 of the suture 64 may be grasped, pulled securely around the ligament and securely tied. When the Nichols-Dechamps suture carrier is used, special care should be given to make sure that the uterine artery is included inside the area encircled by the suture 64.

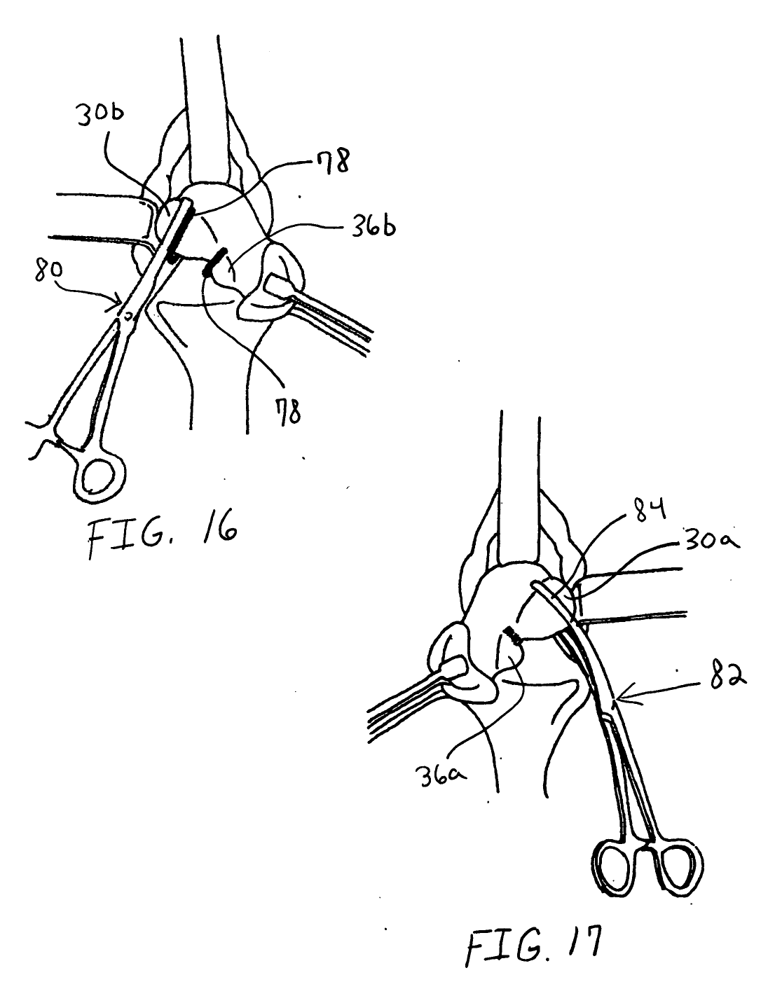

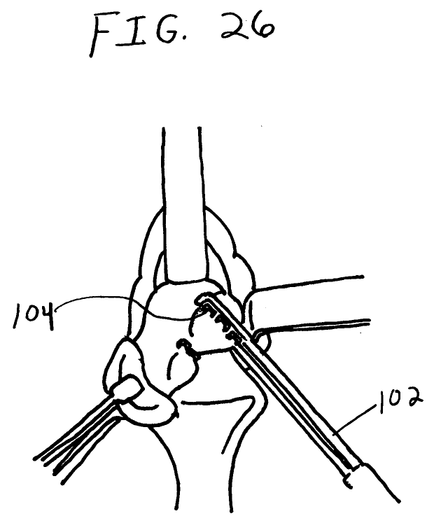

FIG. 16 illustrates an embodiment in which the occlusion is created by compressing the artery and/or ligament by means of a clip 78, which is well known in the art. To compress the artery and/or the ligament, the clip applicator 80 device is inserted on opposite sides of the artery and/or ligament. By squeezing the clip 78 from opposite directions toward one another the artery and/or ligament located in between is compressed. In FIG. 26, a stapling device 102 and staples 104, which are well known in the art, are illustrated. The staples 104 can compress the artery and/or the ligament in a manner similar to the clips 78 already described. In these embodiments use of absorbable staples 104 and/or clips 78 known in the art or to be discovered are preferred. While the use of metal or other rigid staples 104 and/or clips 78 would be successful in compressing the artery and/or ligament, care should be given to ensure that the rigid staple 104 and/or clip 78 does not perforate or otherwise damage the surrounding tissues during activity of the patient, which might include, but would not be limited to, sexual intercourse, exercise, child birth and the like.

Referring now to FIG. 17 a clamping device 82 is illustrated to occlude the ligament and/or artery. As seen in this illustration, the clamp, which is well known in the art, can cause occlusion by crushing the artery and/or ligament. The crushing is accomplished by squeezing the prongs 84 of the device toward each other with the artery and/or ligament located in between.

In other embodiments, the artery and/or blood vessels supplying blood to the uterus 20 can be occluded by cauterization and/or coagulation. When the blood 32 is cauterized and/or coagulated, which generally occurs from the application of heat or energy to the blood, the proteins in the blood are destroyed and, in effect, are turned into cooked proteins. It is to be understood that any manner currently known or to be discovered by which cauterization and/or coagulation causes occlusion of the blood 32 flowing to uterus 20 through the uterine artery 30 and/or the uterosacral ligament 36 is intended to be within the scope of the current invention.

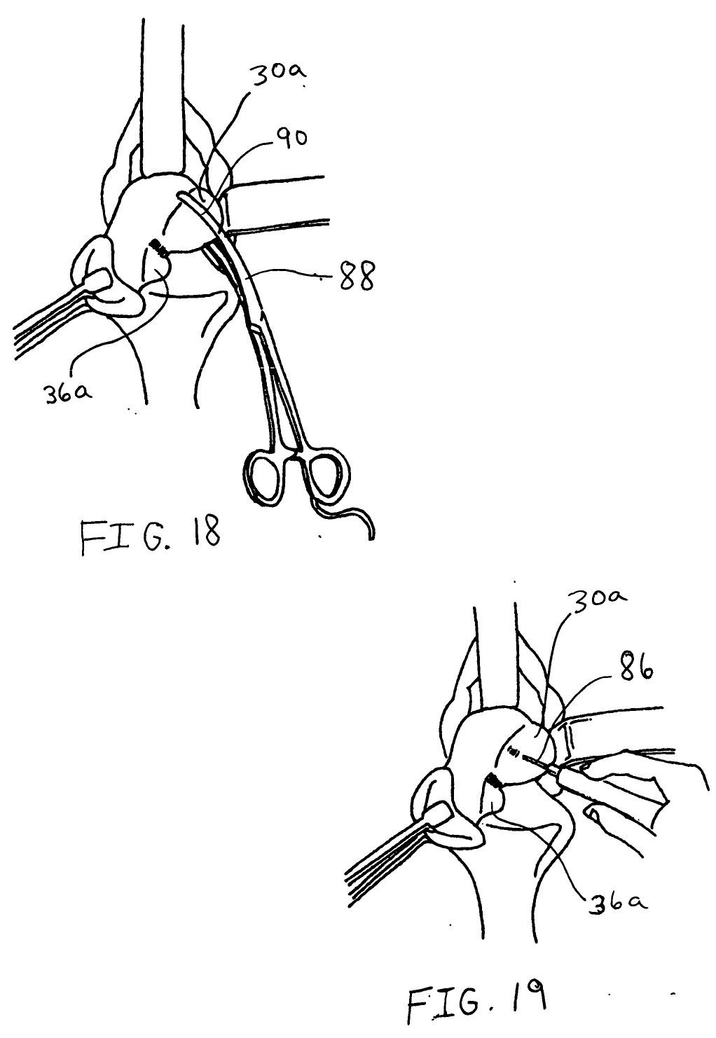

FIG. 18 illustrates a cauterizing device 88, which is well-known in the art. The prongs 90 of the cauterizing device are heated. When the heated prongs 90 impact the ligament and/or artery, the heat is transferred to the blood 32, and the blood coagulates.

FIG. 19 illustrates the use of a laser 86, which is well known in the art to cauterize and/or coagulate the blood in the uterine artery 30. Similarly, the laser 86 could cauterize and/or coagulate the sources of blood flowing through the uterosacral ligament 36 to the uterus 20.

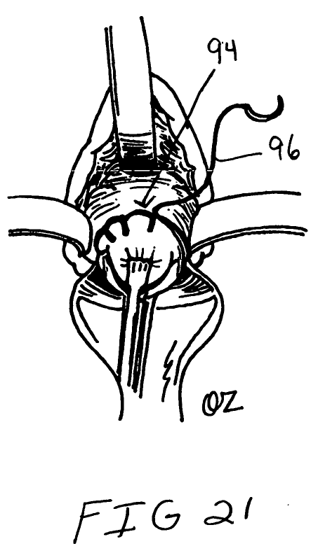

After the blood flow through at least one uterine artery, and optionally through at least one uterosacral ligament 36 is occluded, the next step in the procedure could be to take a measurement of the amount of blood flowing from the uterine artery to the uterus 20. It is to be understood, that while helpful, this step is discretionary. As seen in FIG. 20, a Doppler ultrasound waveform measurement 92 may be recorded of the decrease of blood flow following occlusion. As seen in the example illustrated in FIGS. 4 and 20, use of the Doppler ultrasound has yielded excellent results. However, it is to be understood that other means currently known or to be discovered are intended to be within the scope of the current invention.

As previously described, and by way of example and not intending to be limiting, at this point if the blood flow to the uterus 20 has not been sufficiently reduced, and if the blood 32 flowing through the uterosacral ligaments 36 has not been occluded, the physician could make the decision to occlude the at least one uterosacral ligament 36. Alternatively, as this point if the blood flow to the uterus 20 has been sufficiently reduced, and if the blood flowing through the uterosacral ligaments 36 has not been occluded, the physician could make the decision not to occlude the at least one uterosacral ligament 36.

Referring now to FIG. 21, the next step of the method is to approximate 94 the cervical incision 48. While these Figures illustrate the use of sutures 96, it is to be noted that this step can be accomplished by any of the techniques known in the art or to be discovered to approximate 94 the incision.

Although the invention has been illustrated by reference to specific embodiments, it will be apparent, to those of ordinary skill in the art that various changes and modifications may be made which clearly fall within the scope of the invention. The invention is intended to be protected broadly within the spirit and scope of the appended claims.

1. A method for treating uterine leiomyomas, said method comprising:
vaginally accessing the cardinal ligament surrounding at least one uterine artery, and occluding the flow of blood through said at least one uterine artery.

2. The method of claim 1, said method further comprising occluding the flow of blood through said at least one uterine artery by compressing said at least one artery.

3. The method of claim 2, said method further comprising ligating said cardinal ligament.

4. The method of claim 3, said method further comprising ligating at least one uterosacral ligament.

5. The method of claim 2, said method further comprising occluding the flow of blood through said at least one uterine artery by affixing a clip around at least a portion of said cardinal ligament.

6. The method of claim 2, said method further comprising occluding the flow of blood through said at least one uterine artery by stapling said cardinal ligament.

7. The method of claim 2, said method further comprising occluding the flow of blood through said at least one uterine artery by clamping said cardinal ligament.

8. The method of claim 1, said method further comprising occluding the flow of blood through said at least one uterine artery by coagulating said at least one artery.

9. The method of claim 8, said method further comprising coagulating said at least one artery with a laser.

10. The method of claim 8, said method further comprising coagulating said at least one artery with a cauterizing device.

11. The method of claim 1, said method further comprising occluding the flow of blood through two uterine arteries.

12. The method of claim 1, said method further comprising entering the cul-de-sac and the avascular vesicouterine space.

13. The method of claim 1, said method further comprising measuring blood flow through said uterine artery into said uterus after occluding said at least one artery.

14. A system for treating uterine leiomyomas, said system comprising:
vaginally accessing the cardinal ligament surrounding at least one uterine artery using a surgical suture passing device to extend a ligature around said ligament, and affixing said ligature around said cardinal ligament to occlude the flow of blood through said at least one uterine artery.

15. The system of claim 14, said system further comprising using a surgical suture passing device that has an handle comprised of a thumb and a finger receptacle.

16. The system of claim 14, said system further comprising using a surgical suture passing device that has a jaw portion, said jaw portion being large enough to fit around said cardinal ligament.

17. The system of claim 14, said system further comprising using said surgical suture passing device to ligate at least one uterosacral ligament.

18. A system for treating uterine leiomyomas, said system comprising:
vaginally accessing the cardinal ligament surrounding at least one uterine artery, providing an absorbable clip, and
affixing said clip around said ligament to occlude the flow of blood through said at least one uterine artery.

19. A system for treating uterine leiomyomas, said system comprising:
vaginally accessing the cardinal ligament surrounding at least one uterine artery, providing an absorbable staple, and
affixing said staple around said ligament to occlude the flow of blood through said at least one uterine artery.

20. A system for treating uterine leiomyomas, said system comprising:
vaginally accessing the cardinal ligament surrounding at least one uterine artery, providing a cauterizing device, and
using said cauterizing device to occlude the flow of blood through said at least one uterine artery.

21. A system for treating uterine leiomyomas, said system comprising:
vaginally accessing the cardinal ligament surrounding at least one uterine artery, providing a laser, and
using said laser to occlude the flow of blood through said at least one uterine artery.
